(19) United States
(12) Patent Application Publication
(10) Pub. No.: US 2025/0134471 A1
(43) Pub. Date: May 1, 2025

(54) SYSTEMS AND METHODS FOR CONSTRUCTING A CAPNOGRAM

(71) Applicant: Becton, Dickinson and Company, Franklin Lakes, NJ (US)

(72) Inventors: Nathan Alexander Angel, Oceanside, CA (US); Zhongping Jian, Irvine, CA (US); Feras Al Hatib, Irvine, CA (US)

(21) Appl. No.: 18/933,826

(22) Filed: Oct. 31, 2024

Related U.S. Application Data
(60) Provisional application No. 63/594,894, filed on Oct. 31, 2023.

(51) Int. Cl. A61B 5/00; A61B 5/0205; A61B 5/021; A61B 5/0245; A61B 5/0295; A61B 5/08; G16H 50/70

(52) U.S. Cl. CPC A61B 5/7278; A61B 5/0205; A61B 5/725; A61B 5/7264; A61B 5/742; A61B 5/02108; A61B 5/0245; A61B 5/0295; A61B 5/08; G16H 50/70

(57) ABSTRACT

Systems and methods identify respiration signals in a patient, which can be important for monitoring respiratory health. A respiration signal can be extracted based on data within a physiological waveform, such as a blood pressure waveform, a blood flow waveform, an electrocardiogram, or a plethysmogram. The physiological waveform can be filtered to identify and extract the respiration signal, which can be utilized to construct a capnogram waveform. A respiration rate can be calculated from the respiration signal or from the constructed capnogram waveform.

### CROSS REFERENCE TO RELATED APPLICATIONS

The current application claims the benefit of and priority to U.S. Provisional Patent Application No. 63/594,894 entitled “Systems and Methods for Constructing a Capnogram” filed Oct. 31, 2023, the disclosure of which is hereby incorporated by reference in its entirety for all purposes.

### TECHNOLOGICAL FIELD

The disclosure is generally directed to systems and methods for constructing a capnogram from a physiological waveform.

### BACKGROUND

During critical care situations, including intensive care, surgery, etc., many physiological parameters are important to monitor as indicators of a person's health status. Such parameters include heart rate, blood pressure, blood oxygenation, and respiration. Comprehensive monitoring can require specific equipment and sensors for each of the parameters, which can be burdensome for a patient or can congest a medical facility (e.g., emergency room, surgical theater, operating room, etc.).

Respiration is an important parameter to monitor as indicates whether a patient is breathing at a healthy rate. One common method of measuring respiration is via capnography, which is the monitoring of carbon dioxide (CO₂) levels. Capnography can be performed by a number of methodologies. Generally, the partial pressure of CO₂ of expired breathe is measured using a gas analyzer using a mainstream technique (i.e., CO₂ sensor within airway path) or a sidestream technique (i.e., expiration diverted to CO₂ sensor from airway path). The measurement of CO₂ levels provides a real-time capnogram (i.e., a capnography waveform).

FIG. 1 illustrates an example of a capnogram 100 as a function of partial pressure of carbon dioxide (pCO₂) over time. In capnogram 100, regions are defined inhalation (or inspiration) 102 and exhalation (or expiration) 104, where expiration 104 causes an increase in pCO₂ at a sensor, while inspiration 102 causes a reduction in pCO₂ at a sensor. Expiration 104 can further be divided into different phases. Phase I is the beginning of expiration and represents CO₂ transported into the lungs and occupies space is not involved in gas exchange (i.e., anatomical dead space of respiratory tract). Phase I is mostly indiscernible from the inspiratory phase immediately preceding. Phase II represents CO₂ within the lungs that is forced up the respiratory tract on its way to leave the body, which causes mixing of the air from the dead space with the air in the functional alveoli responsible for gas exchange. Phase III represents the alveolar expiratory flow (i.e., CO₂ derived from the alveoli and not the dead space). The end-tidal carbon dioxide (ETCO₂) is the peak of Phase III and represents the end of expiration.

### SUMMARY

This summary is meant to provide some examples and is not intended to be limiting of the scope of the invention in any way. For example, any feature included in an example of this summary is not required by the claims, unless the claims explicitly recite the features. Various features and steps as described elsewhere in this disclosure may be included in the examples summarized here, and the features and steps described here and elsewhere can be combined in a variety of ways.

In some aspects, the techniques described herein relate to a computational method to construct a capnogram from a physiological waveform.

In some aspects, the techniques described herein relate to a computational method to construct a capnogram from a physiological waveform including: obtaining, utilizing a computational processing system, a physiological waveform.

In some aspects, the techniques described herein relate to a computational method to construct a capnogram from a physiological waveform including: filtering, utilizing the computational processing system, the physiological waveform to yield a respiration waveform.

In some aspects, the techniques described herein relate to a computational method to construct a capnogram from a physiological waveform including: constructing, using the computational processing system, a capnogram based on the respiration waveform.

In some aspects, the techniques described herein relate to a method, wherein obtaining a physiological waveform includes capturing physiological signals from a sensor, wherein the physiological signals are utilized to generate the physiological waveform via the computational processing system.

In some aspects, the techniques described herein relate to a method, wherein the sensor is one of: a blood pressure transducer catheter, a blood pressure cuff, an ultrasound transducer, a MRI scanner, ECG leads, or a PPG.

In some aspects, the techniques described herein relate to a method, wherein the sensor is an invasive sensor.

In some aspects, the techniques described herein relate to a method, wherein the sensor is a noninvasive sensor.

In some aspects, the techniques described herein relate to a method, wherein filtering the physiological waveform and constructing a capnogram are performed while obtaining a physiological waveform.

In some aspects, the techniques described herein relate to a method, wherein a medical monitoring system includes or is in communication with the computational processing system and the sensor.

In some aspects, the techniques described herein relate to a method, wherein obtaining a physiological waveform includes capturing physiological signals from multiple sensors from multiple locations; wherein the physiological signals from the multiple sensors are combined to generate the physiological waveform via the computational processing system.

In some aspects, the techniques described herein relate to a method, wherein the physiological waveform is a blood pressure waveform, a signal proportional to a blood pressure waveform, or a signal derived from a blood pressure waveform.

In some aspects, the techniques described herein relate to a method, wherein the physiological waveform is a blood flow waveform, a signal proportional to a blood flow waveform, or a signal derived from a blood flow waveform.

In some aspects, the techniques described herein relate to a method, wherein the physiological waveform is an electrocardiogram, a signal proportional to an electrocardiogram, or a signal derived from an electrocardiogram.

In some aspects, the techniques described herein relate to a method, wherein the physiological waveform is a plethysmogram, a signal proportional to a plethysmogram, or a signal derived from a plethysmogram.

In some aspects, the techniques described herein relate to a method, wherein filtering the physiological waveform includes using a lowpass filter.

In some aspects, the techniques described herein relate to a method, wherein the lowpass filter has a cutoff frequency that is below a heart rate within the physiological waveform.

In some aspects, the techniques described herein relate to a method, wherein filtering the physiological waveform includes: determining, using the computational processing system, a heart rate from the physiological waveform; and setting, using the computational processing system, a lowpass filter cutoff at a frequency less than the heart rate determined from the physiological waveform.

In some aspects, the techniques described herein relate to a method, wherein the lowpass filter has a cutoff frequency at or below 1 Hz.

In some aspects, the techniques described herein relate to a method, wherein the lowpass filter has a cutoff frequency at or below 0.5 Hz.

In some aspects, the techniques described herein relate to a method, wherein constructing a capnography waveform includes: selecting, using the computational processing system, mathematical bases of capnogram waveform morphologies; and constructing, using the computational processing system, a capnography waveform cycle using the selected mathematical bases and the respiration waveform.

In some aspects, the techniques described herein relate to a method, wherein the mathematical bases are selected from a database of mathematical bases that represent capnogram waveform morphologies.

In some aspects, the techniques described herein relate to a method, wherein selecting mathematical bases is based on clinical data, patient demographic data, or hemodynamic features.

In some aspects, the techniques described herein relate to a method, wherein a sparse number of mathematical bases is selected.

In some aspects, the techniques described herein relate to a method, wherein the number of mathematical bases to be selected can be determined using an equation to identify the bases with a minimum weight or weights.

In some aspects, the techniques described herein relate to a method, wherein a weighting vector is solved to be sparse using L1 regularization or L2 regularization.

In some aspects, the techniques described herein relate to a method, wherein selecting the mathematical bases includes:

$${min}_{w} ( {\sum }_{n = 1}^{N} {w}_{n} {Basis}_{n} - respiration signal ) ,$$

wherein wₙ is a weight of Basisₙ.

In some aspects, the techniques described herein relate to a method, wherein the mathematical bases and respiration waveform are dynamically time warped (DTW) prior to determining the weights for the mathematical bases.

In some aspects, the techniques described herein relate to a method, wherein constructing the capnography waveform cycle includes: Σₙ₌₁^{N} wₙ Basisₙ.

In some aspects, the techniques described herein relate to a method, wherein selecting mathematical bases of capnogram waveform morphologies and constructing the capnography waveform cycle is repeated for each respiration cycle.

In some aspects, the techniques described herein relate to a method further including displaying the capnogram on a display screen, wherein the computational processing system is in connection with the display screen.

In some aspects, the techniques described herein relate to a method further including generating, using the computational processing system, an alert based on the capnogram.

In some aspects, the techniques described herein relate to a method, wherein the alert is to indicate that closer monitoring or a medical intervention should be performed.

In some aspects, the techniques described herein relate to a medical monitoring system for constructing a capnogram.

In some aspects, the techniques described herein relate to a medical monitoring system for constructing a capnogram, including: a sensor, a computational processing system, and a set of instructions stored in memory or in non-transitory media.

In some aspects, the techniques described herein relate to a medical monitoring system for constructing a capnogram, wherein the sensor is capable of capturing physiological signals, and the sensor is in connection with the computational processing system such that physiological signal data captured by the sensor can be transmitted to the computational processing system.

In some aspects, the techniques described herein relate to a medical monitoring system for constructing a capnogram, wherein the set of instructions direct the computational processing system to: receive a physiological waveform from the physiological sensor data derived from the sensor.

In some aspects, the techniques described herein relate to a medical monitoring system for constructing a capnogram, wherein the set of instructions direct the computational processing system to: filter the physiological waveform to yield a respiration waveform.

In some aspects, the techniques described herein relate to a medical monitoring system for constructing a capnogram, wherein the set of instructions direct the computational processing system to: construct a capnogram based on the respiration waveform.

In some aspects, the techniques described herein relate to a system, wherein the capnogram is constructed as the physiological sensor data is being received.

In some aspects, the techniques described herein relate to a system, wherein the sensor is one of: a blood pressure transducer catheter, a blood pressure cuff, an ultrasound transducer, a MRI scanner, ECG leads, or a PPG.

In some aspects, the techniques described herein relate to a system, wherein the sensor is an invasive sensor.

In some aspects, the techniques described herein relate to a system, wherein the sensor is a noninvasive sensor.

In some aspects, the techniques described herein relate to a system wherein the set of instructions further direct the computational processing system to: receive physiological sensor data from the sensor; and generate the physiological waveform from the physiological sensor data received from the data sensor.

In some aspects, the techniques described herein relate to a system including two or more sensors, wherein the set of instructions further direct the computational processing system to: receive physiological sensor data from each of the two or more sensors; and generate the physiological waveform from the physiological sensor data received from the data two or more sensors; wherein the physiological sensor data from each sensor is combined to generate the physiological waveform.

In some aspects, the techniques described herein relate to a system, wherein the physiological waveform is a blood pressure waveform, a signal proportional to a blood pressure waveform, or a signal derived from a blood pressure waveform.

In some aspects, the techniques described herein relate to a system, wherein the physiological waveform is a blood flow waveform, a signal proportional to a blood flow waveform, or a signal derived from a blood flow waveform.

In some aspects, the techniques described herein relate to a system, wherein the physiological waveform is an electrocardiogram, a signal proportional to an electrocardiogram, or a signal derived from an electrocardiogram.

In some aspects, the techniques described herein relate to a system, wherein the physiological waveform is a plethysmogram, a signal proportional to a plethysmogram, or a signal derived from a plethysmogram.

In some aspects, the techniques described herein relate to a system, wherein a lowpass filter is used to filter the physiological waveform.

In some aspects, the techniques described herein relate to a system, wherein the lowpass filter has a cutoff frequency that is below a heart rate within the physiological waveform.

In some aspects, the techniques described herein relate to a system, wherein the set of instructions further direct the computational processing system to: determine a heart rate from the physiological sensor data; and set a lowpass filter cutoff at a frequency less than the heart rate determined from the physiological signals.

In some aspects, the techniques described herein relate to a system, wherein the lowpass filter has a cutoff frequency at or below 1 Hz.

In some aspects, the techniques described herein relate to a system, wherein the lowpass filter has a cutoff frequency at or below 0.5 Hz.

In some aspects, the techniques described herein relate to a system, wherein the set of instructions further direct the computational processing system to: select mathematical bases of capnogram waveform morphologies; and construct a capnography waveform cycle using the selected mathematical bases and the respiration waveform.

In some aspects, the techniques described herein relate to a system, wherein a database of mathematical bases that represent capnogram waveform morphologies is utilized for selecting the mathematical bases.

In some aspects, the techniques described herein relate to a system, wherein the database of mathematical bases that represent capnogram waveform morphologies is stored in the memory or in the non-transitory media.

In some aspects, the techniques described herein relate to a system, wherein selecting mathematical bases is based on clinical data, patient demographic data, or hemodynamic features.

In some aspects, the techniques described herein relate to a system, wherein a sparse number of mathematical bases is selected.

In some aspects, the techniques described herein relate to a system, wherein the number of mathematical bases to be selected can be determined using an equation to identify the bases with a minimum weight or weights.

In some aspects, the techniques described herein relate to a system, wherein a weighting vector is solved to be sparse using L1 regularization or L2 regularization.

In some aspects, the techniques described herein relate to a system, wherein:

$${min}_{w} ( {\sum }_{n = 1}^{N} {w}_{n} {Basis}_{n} - respiration signal )$$

is utilized to select the mathematical bases; wherein wₙ is a weight of Basisₙ.

In some aspects, the techniques described herein relate to a system, wherein the mathematical bases and respiration waveform are dynamically time warped (DTW) prior to determining the weights for the mathematical bases.

In some aspects, the techniques described herein relate to a system, wherein: Σₙ₌₁^{N} wₙ Basisₙ is utilized to construct the capnography waveform cycle.

In some aspects, the techniques described herein relate to a system, wherein selection of the mathematical bases of capnogram waveform morphologies and construction of the capnography waveform cycle are repeated for each respiration cycle.

In some aspects, the techniques described herein relate to a system further including a display screen in connection with the computational processing system; wherein the set of instructions further direct the computational processing system to display the capnogram on a display screen.

In some aspects, the techniques described herein relate to a system, wherein the set of instructions further direct the computational processing system to generate an alert based on the capnogram.

In some aspects, the techniques described herein relate to a system, wherein the alert is to indicate that closer monitoring or a medical intervention should be performed.

In some aspects, the techniques described herein relate to a system, wherein the medical monitoring system is a hemodynamic monitoring system or an electrocardiogram system.

In some aspects, the techniques described herein relate to a computational method for performing a pulmonary capillary wedge pressure (PCWP) measurement of an individual.

In some aspects, the techniques described herein relate to a computational method for performing a pulmonary capillary wedge pressure (PCWP) measurement of an individual including: acquiring a blood pressure waveform of an individual.

In some aspects, the techniques described herein relate to a computational method for performing a pulmonary capillary wedge pressure (PCWP) measurement of an individual, wherein the waveform includes a blood pressure measurement acquired from a pulmonary artery position and a blood pressure measurement acquired from a wedge position.

In some aspects, the techniques described herein relate to a computational method for performing a pulmonary capillary wedge pressure (PCWP) measurement of an individual, wherein the blood pressure waveform is acquired using a pulmonary artery catheter.

In some aspects, the techniques described herein relate to a computational method for performing a pulmonary capillary wedge pressure (PCWP) measurement of an individual including: constructing, using a computational processing system and the blood pressure waveform, a capnogram of the individual.

In some aspects, the techniques described herein relate to a computational method for performing a pulmonary capillary wedge pressure (PCWP) measurement of an individual including: determining, using a computational processing system, a PCWP measurement from the blood pressure measurement from the wedge position, wherein the PCWP measurement is determined based on the capnogram of the individual.

In some aspects, the techniques described herein relate to a method, wherein the determining of the PCWP includes determining a blood pressure measurement from the wedge position at an end-tidal CO2 peak of the capnogram.

In some aspects, the techniques described herein relate to a method, wherein the determining of the PCWP includes averaging blood pressure measurement from the wedge position across one or more respiratory cycles of the capnogram.

In some aspects, the techniques described herein relate to a method, wherein the PCWP measurement is generated and the capnogram is constructed as the blood pressure waveform is acquired.

In some aspects, the techniques described herein relate to a method further including: displaying the PCWP measurement and the capnogram on a display screen in digital communication with the computational processing system.

In some aspects, the techniques described herein relate to a method, wherein the computational processing system and the pulmonary artery catheter are part of a hemodynamic monitoring system.

In some aspects, the techniques described herein relate to a method, wherein acquiring the blood pressure waveform includes: inserting the pulmonary artery catheter into a central vein of the individual, guiding the pulmonary artery catheter to the pulmonary artery; and inflating a balloon at or near a distal end of the pulmonary artery catheter to allow the pulmonary artery catheter to migrate to the wedge position.

In some aspects, the techniques described herein relate to a hemodynamic monitoring system for performing a pulmonary capillary wedge pressure (PCWP) measurement.

In some aspects, the techniques described herein relate to a hemodynamic monitoring system for performing a pulmonary capillary wedge pressure (PCWP) measurement, including: a pulmonary artery catheter configured to acquire blood pressure measurement acquired from a pulmonary artery position and a blood pressure measurement acquired from a wedge position.

In some aspects, the techniques described herein relate to a hemodynamic monitoring system for performing a pulmonary capillary wedge pressure (PCWP) measurement, wherein the pulmonary artery catheter includes a balloon that is inflatable.

In some aspects, the techniques described herein relate to a hemodynamic monitoring system for performing a pulmonary capillary wedge pressure (PCWP) measurement, including a computational processing system in connection with the pulmonary artery catheter, the computational processing system including: a processor system; a display screen in digital connection with the processor system; and a memory system including one or more applications that can direct the processor system.

In some aspects, the techniques described herein relate to a hemodynamic monitoring system for performing a pulmonary capillary wedge pressure (PCWP) measurement, wherein the one or more applications that can direct the processor system to: acquire a blood pressure measurement in a pulmonary artery position.

In some aspects, the techniques described herein relate to a hemodynamic monitoring system for performing a pulmonary capillary wedge pressure (PCWP) measurement, wherein the one or more applications that can direct the processor system to: inflate the balloon, wherein inflating the balloon allows the catheter to migrate to a wedge position.

In some aspects, the techniques described herein relate to a hemodynamic monitoring system for performing a pulmonary capillary wedge pressure (PCWP) measurement, wherein the one or more applications that can direct the processor system to: acquire a blood pressure measurement in the wedge position; generate a blood pressure waveform from blood pressure measurement acquired from a pulmonary artery position and a blood pressure measurement acquired from a wedge position.

In some aspects, the techniques described herein relate to a hemodynamic monitoring system for performing a pulmonary capillary wedge pressure (PCWP) measurement, wherein the one or more applications that can direct the processor system to: construct a capnogram using the blood pressure waveform; determine a PCWP measurement from the blood pressure measurement from the wedge position, wherein the PCWP measurement is determined based on the capnogram.

In some aspects, the techniques described herein relate to a hemodynamic monitoring system for performing a pulmonary capillary wedge pressure (PCWP) measurement, wherein the one or more applications that can direct the processor system to: display one or more of: the blood pressure waveform, the PCWP measurement, or the capnogram on the display screen.

In some aspects, the techniques described herein relate to a system, wherein the determining of the PCWP includes determining a blood pressure measurement from the wedge position at an end-tidal CO2 peak of the capnogram.

In some aspects, the techniques described herein relate to a system, wherein the determining of the PCWP includes averaging blood pressure measurement from the wedge position across one or more respiratory cycles of the capnogram.

In some aspects, the techniques described herein relate to a system, wherein the blood pressure waveform is generated and displayed on the display screen in real time.

In some aspects, the techniques described herein relate to a system, wherein the PCWP measurement is determined and displayed on the display screen in real time.

In some aspects, the techniques described herein relate to a system, wherein the capnogram is constructed and displayed on the display screen as the blood pressure waveform is generated.

Other features and advantages of the present invention will become apparent from the following detailed description, taken in conjunction with the accompanying drawings which illustrate, by way of example, the principles of the invention.

### BRIEF DESCRIPTION OF THE DRAWINGS

The description and claims will be more fully understood with reference to the following figures and data graphs, which are presented as exemplary embodiments of the invention and should not be construed as a complete recitation of the scope of the invention.

FIG. 1 illustrates an example of a respiration signal (or capnogram) as a function of partial pressure of carbon dioxide (pCO₂) over time.

FIG. 2 illustrates an example of a blood pressure waveform plotted as a function of pressure (P) over time (t).

FIG. 3A illustrates an example of a blood pressure signal comprising individual beats with respiration cycles overlaid.

FIG. 3B illustrates an example of a respiration waveform with a capnogram overlaid.

FIG. 4 illustrates an example of a capnography constructed from a blood pressure signal and is compared to traditionally acquired capnography and a respiration waveform.

FIG. 5 illustrates an example of a flow chart for identifying a respiration signal from a physiological waveform.

FIG. 6 illustrates an example of a medical monitoring systems.

FIG. 7 illustrates an example of a distributed computing system for medical monitoring.

FIG. 8 illustrates an example of performing computer-assisted pulmonary capillary wedge pressure utilizing a constructed capnogram.

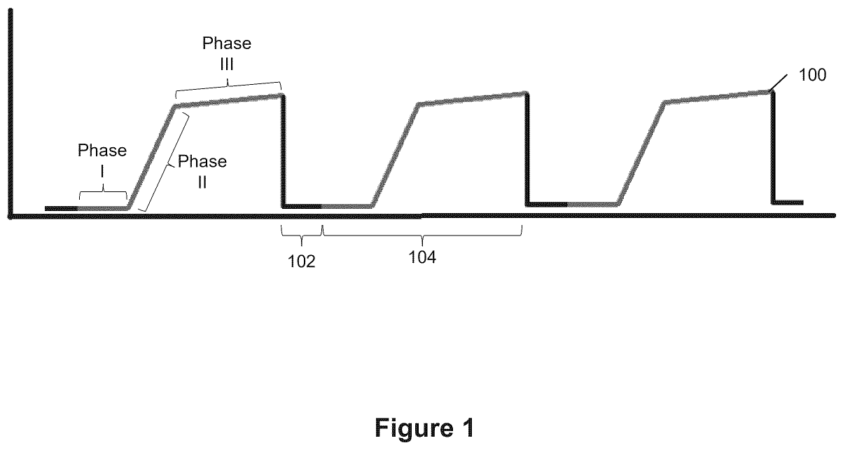

### DETAILED DESCRIPTION

The current description provides systems and methods for monitoring respiration of an individual, which can be monitored without direct measurement of gaseous components. In several implementations, respiration is monitored by generating a capnogram that is derived from physiological waveform. To generate a capnogram, a lowpass filter with a frequency can be utilized, where the lowpass filter is less than or equal to the frequency of a heart rate.

When a patient is in a medical facility, their rate of respiration is important to monitor as it can provide indication of various health conditions and disorders. Capnograms can provide a reliable of index of effective heart compression during CPR, help manage endotracheal intubation, and assessment of respiratory depression under procedural sedation and analgesia. Capnograms can also help diagnose the presence of obstructive pulmonary disease, a pulmonary embolism, heart failure, hypotensive shock, metabolic disorder, and severe traumatic injury.

Typical methods to generate a capnogram utilize a gas analyzer in a mainstream technique or a sidestream technique. Gas analysis often requires that the patient be intubated and/or secured to a mask to capture expiration. It would be ideal to expand the use of capnograms by utilizing alternative techniques by generating capnograms from other physiological sensors, which may be easier to use, more readily accessible, and/or already being utilized for other physiological assessments. Accordingly, the current disclosure describes various systems and methods that generate capnogram from a physiological waveform. Various physiological waveforms and sensors to construct a capnogram can be utilized, including (but not limited to) a blood pressure waveform, a blood flow waveform, an electrocardiogram (ECG), and a plethysmogram. In some implementations, the physiological waveform to construct a capnogram comprises a blood pressure waveform, a signal proportional to a blood pressure waveform, or a signal derived from a blood pressure waveform. In some implementations, the physiological waveform to construct a capnogram comprises a blood flow waveform, a signal proportional to a blood flow waveform, or a signal derived from a blood flow waveform. In some implementations, the physiological waveform to construct a capnogram comprises an electrocardiogram, a signal proportional to an electrocardiogram, or a signal derived from an electrocardiogram. In some implementations, the physiological waveform to construct a capnogram comprises a plethysmogram, a signal proportional to a plethysmogram, or a signal derived from a plethysmogram.

Physiological waveforms can be generated or derived from physiological signals derived from invasive and/or noninvasive sensors. Blood pressure waveforms can be captured utilizing (or derived from) a blood pressure transducer catheter (e.g., intra-arterial catheters, pulmonary wedge catheters, cardiac catheters, and intravenous catheters), a blood pressure cuff, and an ultrasound transducer. Blood flow waveforms can be captured utilizing (or derived from) an ultrasound transducer and magnetic resonance imaging (MRI) scanner. ECGs can be captured utilizing (or derived from) one or more leads (i.e., electrodes) in connection with an electrocardiogramachine. Plethysmograms can be captured utilizing (or derived from) any plethysmography technique to measure blood volume, such using a photoplethysmograph for optical transmission and reflection of an artery or vein. A photoplethysmograph can be utilized within blood pressure cuffs for continuous blood pressure monitoring.

Physiological waveforms can also be computed by various methods. For example, a blood flow waveform can be computed from a blood pressure waveform. In some instances, a model of input impedance of pulmonary circulation is used to compute a blood flow waveform, which can be computed using (for example) the physiological Windkessel model.

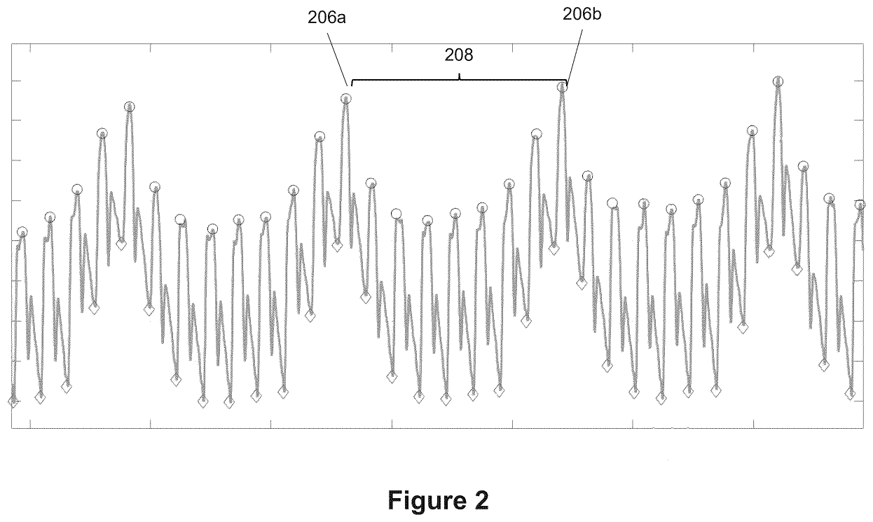

Provided in FIG. 2 is an example of a blood pressure waveform 200 plotted as a function of pressure (P) over time (1), which can be captured invasively or noninvasively utilizing sensors as described herein. Blood pressure waveform 200 comprises several continuous and rhythmic heartbeats 202, which are identifiable as time between diastolic minima pressures 204 (minima for each beat 202). Each beat 202 is further characterized by a systolic peak pressures 206. Because heartbeats are repeated cycles, a heartbeat can be defined as time between any landmark of the blood pressure waveform (e.g., time between systolic pressures 206, time between dicrotic notches, etc.).

To construct a capnogram, lowpass filtering of a physiological waveform can be performed. As can be seen in FIG. 2, blood pressure waveform 200 has a cyclical variation that arises due to respiration, as exemplified by cycle 208 between two systolic peak pressure maxima 206a and 206b. The respiration-related cycles can be used as a template for constructing the capnogram. To do so, a lowpass filter with a cutoff at or below the heart rate frequency can reveal a respiratory cycle. Because an average heart rate is typically 60 beats per minute (bpm) or greater, a filter cutoff at or below 1 hertz (i.e., 1 beat per second) will remove individual heartbeats to reveal the respiratory pattern. Filtering can be performed with a finite impulse response filter or an infinite impulse response filter.

Any filter cutoff below or equal to a heart rate of an individual can be utilized. Typically, human heart rate ranges between 50 bpm up to 200 bpm and thus a lowpass filter cutoff for human can be any cutoff within range from about 0.83 Hz up to about 3.3 Hz. In some implementations, the lowpass filter cutoff is at or below 1 Hz. In some implementations, the lowpass filter cutoff is at or below 0.5 Hz. It should be understood that a lowpass filter cutoff can be lower, which may provide a more smoothened respiratory curve.

In various implementations, a lowpass filter cutoff is less than 0.15 Hz, between 0.10 Hz and 0.20 Hz, between 0.15 Hz and 0.25 Hz, between 0.20 Hz and 0.30 Hz, between 0.25 Hz and 0.35 Hz, between 0.30 Hz and 0.40 Hz, between 0.35 Hz and 0.45 Hz, between 0.40 Hz and 0.50 Hz, between 0.45 Hz and 0.55 Hz, between 0.50 Hz and 0.60 Hz, between 0.55 Hz and 0.65 Hz, between 0.60 Hz and 0.70 Hz, between 0.65 Hz and 0.75 Hz, between 0.70 Hz and 0.80 Hz, between 0.75 Hz and 0.85 Hz, between 0.80 Hz and 0.90 Hz, between 0.85 Hz and 0.95 Hz, between 0.90 Hz and 1.00 Hz, between 0.95 Hz and 1.05 Hz, between 1.00 Hz and 1.10 Hz, between 1.05 Hz and 1.15 Hz, between 1.10 Hz and 1.20 Hz, between 1.15 Hz and 1.25 Hz, between 1.20 Hz and 1.30 Hz, between 1.25 Hz and 1.35 Hz, between 1.30 Hz and 1.40 Hz, between 1.35 Hz and 1.45 Hz, between 1.40 Hz and 1.50 Hz, between 1.45 Hz and 1.55 Hz, between 1.50 Hz and 1.60 Hz, between 1.55 Hz and 1.65 Hz, between 1.60 Hz and 1.70 Hz, between 1.65 Hz and 1.75 Hz, between 1.70 Hz and 1.80 Hz, between 1.75 Hz and 1.85 Hz, between 1.80 Hz and 1.90 Hz, between 1.85 Hz and 1.95 Hz, between 1.90 Hz and 2.00 Hz, between 1.95 Hz and 2.05 Hz, between 2.00 Hz and 2.10 Hz, between 2.05 Hz and 2.15 Hz, between 2.10 Hz and 2.20 Hz, between 2.15 Hz and 2.25 Hz, between 2.20 Hz and 2.30 Hz, between 2.25 Hz and 2.35 Hz, between 2.30 Hz and 2.40 Hz, between 2.35 Hz and 2.45 Hz, between 2.40 Hz and 2.50 Hz, between 2.45 Hz and 2.55 Hz, between 2.50 Hz and 2.60 Hz, between 2.55 Hz and 2.65 Hz, between 2.60 Hz and 2.70 Hz, between 2.65 Hz and 2.75 Hz, between 2.70 Hz and 2.80 Hz, between 2.75 Hz and 2.85 Hz, between 2.80 Hz and 2.90 Hz, between 2.85 Hz and 2.95 Hz, between 2.90 Hz and 3.00 Hz, between 3.95 Hz and 3.05 Hz, between 3.00 Hz and 3.10 Hz, between 3.05 Hz and 3.15 Hz, between 3.10 Hz and 3.20 Hz, between 3.15 Hz and 3.25 Hz, between 3.20 Hz and 3.30 Hz, between 3.25 Hz and 3.35 Hz, between 3.30 Hz and 3.40 Hz, between 3.35 Hz and 3.45 Hz, or between 3.40 Hz and 3.50 Hz. In some implementations, a lowpass filter cutoff is determined based on a patient's heart rate. In some implementations, a lowpass filter cutoff is based on a heart rate within the physiological waveform. Accordingly, in some implementations, the heart rate is determined from the physiological signals and the lowpass filter is set to be below the heart rate determined from the physiological signals. In some instances, a lowpass filter cutoff can be determined by patient heart rate and may be adjusted as needed during a procedure.

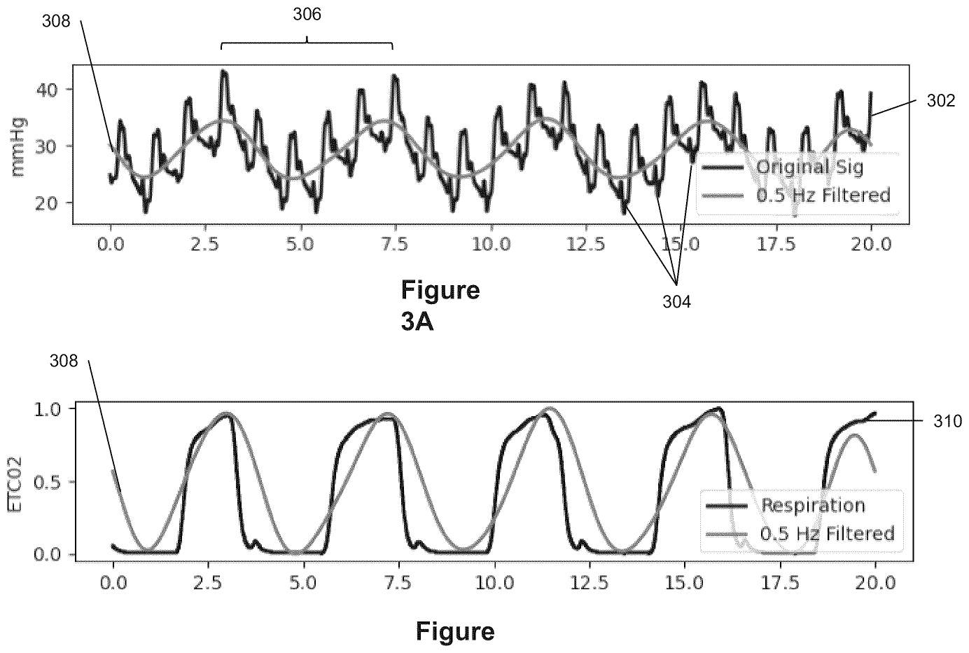

FIG. 3A provides an example of a physiological waveform of a patient that is filtered to yield a respiratory waveform. Blood pressure waveform 302 comprises rhythmic heartbeats 304 that undulate up and down in accordance with respiration cycles 306. The blood pressure waveform 302 was filtered with a lowpass filter with a cutoff of 0.5 Hz to yield respiratory waveform 308 having a generally sinusoidal pattern. The number of cycles of respiratory waveform 308 over time yields a respiration rate for the patient. The respiratory waveform should mimic the cycles as captured by capnography, such as illustrated in the example of FIG. 3B. Specifically, FIG. 3B illustrates respiratory waveform 308 plotted on top of a capnogram 310 of the patient as measured by end-tidal carbon dioxide (ETCO₂). Capnogram 310 can be considered a “ground truth” for respiration; thus, the correlation between peaks in the capnogram 310 and respiration waveform 308 indicate that filtering of a physiological waveform.

Although FIGS. 3A and 3B illustrate low-band filtering of a blood pressure waveform, such a filtering process can be performed on another physiological waveform that has respiration-induced variation. Other waveforms that can be filtered include (but are not limited to) blood flow waveforms, ECG waveforms, and PPG waveforms. Blood flow waveforms can be obtained directly (e.g., from an invasive ultrasound probe) and/or computed from a blood pressure waveform. Such computations can be generated de novo and/or known in the art. In some instances, a model of input impedance of pulmonary circulation is used to compute a blood flow waveform—an exemplary model is the physiological Windkessel model.

Upon generating a respiratory waveform, a capnogram can be constructed based on a respiratory waveform. A capnogram can be constructed by selecting mathematical bases of capnogram waveform morphologies that are representative of capnographs that would be acquired by traditional means (e.g., capnographs generated by measuring CO₂ of expired breath). Mathematical bases of capnogram waveform morphologies can be selected from a database. Each basis in the database can represent a particular variation in waveform morphology. Waveform morphologies that can be selected include (for example) slope of rise, slope of fall, inspiration period, expiration period, and/or any other measurable or observable feature of a capnogram waveform.

In some implementations, a database of mathematical bases that represent capnogram waveform morphologies can be generated using capnographs captured from a collection of patients. In some implementations, mathematical bases that represent capnogram waveform morphologies can be generated de novo by collecting from patients. If a patient has had their capnogram generated and stored, personalized mathematical bases that represent capnogram waveform morphologies of the patient can be utilized. In some implementations, mathematical bases representative of capnogram waveform morphologies can be labeled with certain patient characteristics that are associated with particular morphologies. For example, certain clinical data, patient demographic data, and hemodynamic features are associated with particular morphologies, which may allow for filtering of databases (e.g., filtering database based on patient age) and/or generating more specific databases (e.g., generating a plurality databases specific for particular age ranges). Some clinical data that may be used to to select bases includes whether the patient is under mechanical ventilation or is spontaneously breathing.

In some implementations, to prevent overfitting, a sparse number of selected representative mathematical bases can be selected. Overfitting can result in a capnogram that highly mimics the respiration signal, yielding a waveform that appears sinusoidal rather than generating a waveform with a morphology similar to traditionally generated capnography. Any methodology to limit the number of mathematical bases selected can be utilized. In some implementations, the number of mathematical bases to be selected by limiting the bases available for selection based on clinical data, patient demographic data, or hemodynamic features. In some implementations, the number of mathematical bases to be selected can be determined using a selected number of bases (e.g., the sparse number of bases to be selected is 1, 2, 3, 4, 5, or another number that does not produce a sinusoidal waveform). In some implementations, the number of mathematical bases to be selected can be determined using an equation to identify the bases with the minimum weight or weights. In some implementations, the weighting vector can also be solved to be sparse such as using L1 regularization (LASSO regression) or L2 regularization (Ridge Regression). An exemplary equation to select mathematical bases is Equation 1:

$${min}_{w} ( {\sum }_{n = 1}^{N} {w}_{n} {Basis}_{n} - respiration waveform ) \qquad ( 1 )$$

where wₙ is a weight of Basisₙ. In some implementations, the the mathematical bases and respiration waveform are dynamically time warped (DTW) prior to determining the weights for the basis, which can be reversed back after the capnograph waveform cycle is constructed.

Once the sparse number of mathematical bases are selected, the capnogram can be constructed based on the respiration waveform for each cycle. Such construction can utilize an equation such as Equation 2:

$${\sum }_{n = 1}^{N} {w}_{n} {Basis}_{n} \qquad ( 2 )$$

Accordingly, a cycle of the respiration waveform is derived, a capnogram waveform cycle with an appropriate morphology is then determined from the respiration waveform and selected bases. Upon determining the capnogram waveform cycle, the respiration waveform cycle is replaced with the determined capnogram waveform cycle. The process is repeated for each respiratory cycle such that a continuous capnogram is generated and displayed. The capnogram can be smoothened such that the displayed appears to continuous and/or less segmented.

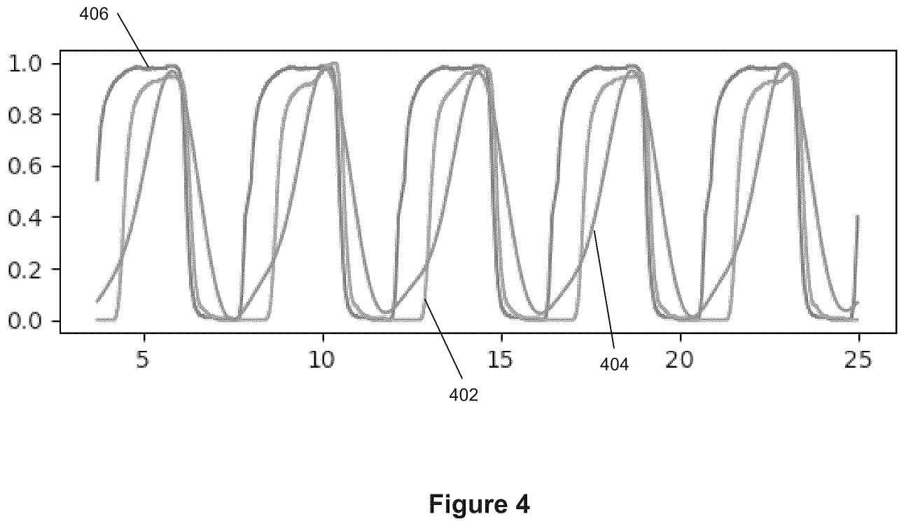

Resulting constructed capnograms are comparable to traditionally acquired capnograms. FIG. 4 provides an example of a constructed capnogram of a patient as compared to a traditionally acquired capnogram and a respiration waveform. Specifically, a normalized capnogram 402 is plotted as a ground-truth respiration signal. A pressure derived respiration waveform 404 is generated by filtering a blood pressure signal. Further, the is plotted showing the correlation to the capnogram 402. Further, a constructed capnograph waveform 406 was constructed using the generated respiration waveform and selected bases. As shown in FIG. 4, the constructed capnograph waveform 406 closely resembles the traditionally acquired capnogram 402. These results show that a constructed capnogram can accurate recapitulate a traditionally acquired capnogram. Accordingly, these results highlight the ability to forgo traditionally acquiring a capnogram and instead construct a capanogram from a blood pressure waveform or another physiological waveform such as a blood flow waveform, an electrocardiogram (ECG), or a plethysmogram waveform.

### Processes for Constructing a Capnogram

Various systems (e.g., computational and/or hemodynamic monitoring systems) can perform a computational process to construct a capnogram from a physiological waveform. The construction of a capnogram may be performed in situations in which a physiological waveform is captured and/or derived from another sensor. In some implementations, the construction of a capnogram is performed without direct measurement of gaseous components.

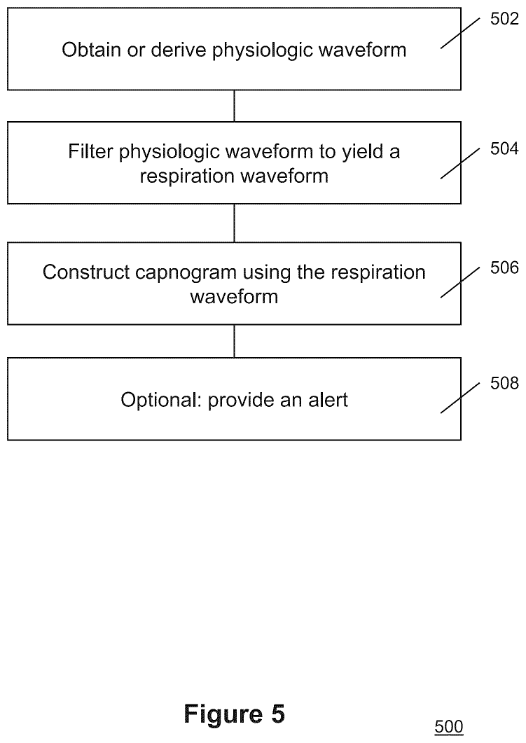

Provided in FIG. 5 is an example of a process that can be performed by a computational system and/or hemodynamic monitoring system to construct a capnogram from a physiological waveform. Process 500 can filter a physiological waveform with a lowpass filter cutoff to yield a respiratory waveform that can be utilized as a template to construct capnogram by constructing capnogram waveform cycles with appropriate morphology using representative mathematical bases.

In process 500, a physiological waveform is obtained or derived for an individual at 502. As described elsewhere herein, the physiological waveform can be blood pressure, blood flow, ECG, PPG, and/or any other physiological signal that exhibits respiration induced variation. Such waveforms can be obtained or derived invasively or non-invasively through the use of sensors such as (for example) a pressure catheter, ultrasound probe, blood pressure cuff, one or more electrodes, a photoplethymograph and/or any other applicable sensor for obtaining or deriving a physiological waveform. Physiological waveforms can be obtained or derived in real-time or from a trace file of a previously obtained or derived collection of physiological signals.

Physiological signals can be derived or obtained using from any applicable location within the cardiovascular system, including (but not limited to) the heart, a peripheral artery (e.g., such as the radial artery, the brachial artery, or the femoral artery), or a capillary (e.g., capillaries of a digit). In one example, the physiological waveform is a blood pressure waveform obtained from a right heart, which can be obtained within the right ventricle, right atrium, pulmonary artery, pulmonary wedge, pulmonary vein, etc.

Physiological signals can be obtained or derived using a continuous or a sampling method. Sampling may be performed based on a time frame (e.g., every 5 seconds) or based on one or more physiological parameters, such as heartbeats and respiration cycles.

Physiological signals can be simultaneously obtained from one or more sensors in one or more locations. In one example, pressure signals can be obtained from a right ventricle and pulmonary artery using an appropriate catheter, such as a Swan-Ganz catheter, which uses multiple lumens to capture pressure signals from multiple locations simultaneously. In another example, pressure signals can be obtained using a noninvasive sensor (e.g, a blood pressure cuff) contemporaneously with an invasive sensor (e.g., radial catheter). Physiological waveforms can be generated by combining multiple signals, as is appreciated in the art.

At 504, a physiological waveform is filtered to yield a respiration waveform. In many implementations, a lowpass filter with a cutoff at or below the heart rate frequency is utilized to filter the physiological waveform. Any filter cutoff below or equal to a heart rate of an individual can be utilized. Typically, human heart rate ranges between 50 bpm up to 200 bpm and thus a lowpass filter cutoff for human can be any cutoff within a range from about 0.83 Hz up to about 3.3 Hz. It should be understood that a lowpass filter cutoff can be lower, which may provide a more smoothened respiratory curve.

At 506, a capnogram is constructed using the respiration waveform. To construct a capnogram, mathematical bases of capnogram waveform morphologies can be selected that are representative of traditionally acquired capnographs. In some implementations, mathematical bases of capnogram waveform morphologies are selected from a database. Each basis in the database can represent a particular variation in waveform morphology. Waveform morphologies that can be selected include (for example) slope of rise, slope of fall, inspiration period, expiration period, and/or any other measurable or observable feature of a capnogram waveform.

In some implementations, to prevent overfitting, a sparse number of selected representative mathematical bases can be selected. Any methodology to limit the number of mathematical bases selected can be utilized. In some implementations, the number of mathematical bases to be selected by limiting the bases available for selection based on clinical data, patient demographic data, or hemodynamic features. In some implementations, the number of mathematical bases to be selected is 1, 2, 3, 4, 5, or another number that does not produce a sinusoidal waveform. In some implementations, the number of mathematical bases to be selected is determined using an equation to identify the bases with the minimum weight or weights. In some implementations, the weighting vector can also be solved to be sparse such as using L1 regularization (LASSO regression) or L2 regularization (Ridge Regression). In some implementations, the number of mathematical bases to be selected is determined using Equation 1.

Upon selecting mathematical bases, the capnogram can be constructed based on the respiration waveform for each cycle. In some instances, a cycle of the respiration waveform is derived, a capnogram waveform cycle with an appropriate morphology is then determined from the respiration waveform and selected bases. Upon determining the capnogram waveform cycle, the respiration waveform cycle is replaced with the determined capnogram waveform cycle. In some instances, the capnogram waveform cycle is constructed utilizing Equation 2. The selection of a capnogram waveform cycle can be repeated for each respiratory cycle such that a continuous capnogram is generated and displayed. In some instances, the capnogram can be smoothened such that the displayed appears to continuous and/or less segmented.

Capnograms can be utilized for various clinical applications, including (but not limited to) assessment airway integrity, prediction of outcomes in intensive care, determination of intraoperative complications, and sedation monitoring. Accordingly, at step 508, the capnogram is optionally utilized to provide an, which can be utilized to indicate that closer monitoring and/or a medical intervention should be performed. For example, if a patient is receiving oxygen, an alert can be provided to suggest that the patient may need an increase or a decrease of oxygen concentration, an increase or a decrease of gas flow rate, and/or any other relevant intervention to modulate, moderate, and/or stabilize a patient respiration.

Important respiration features (such as respiration rate) can be computed either from the respiration signal obtained from the physiological signal or from the constructed capnogram waveform.

It should be noted that features of process 500 can be altered, omitted, repeated, or added to, as appropriate without straying from the contents of this disclosure. In certain instances, certain features may be performed in a different order and/or simultaneously.

### Computer Executed Implementations

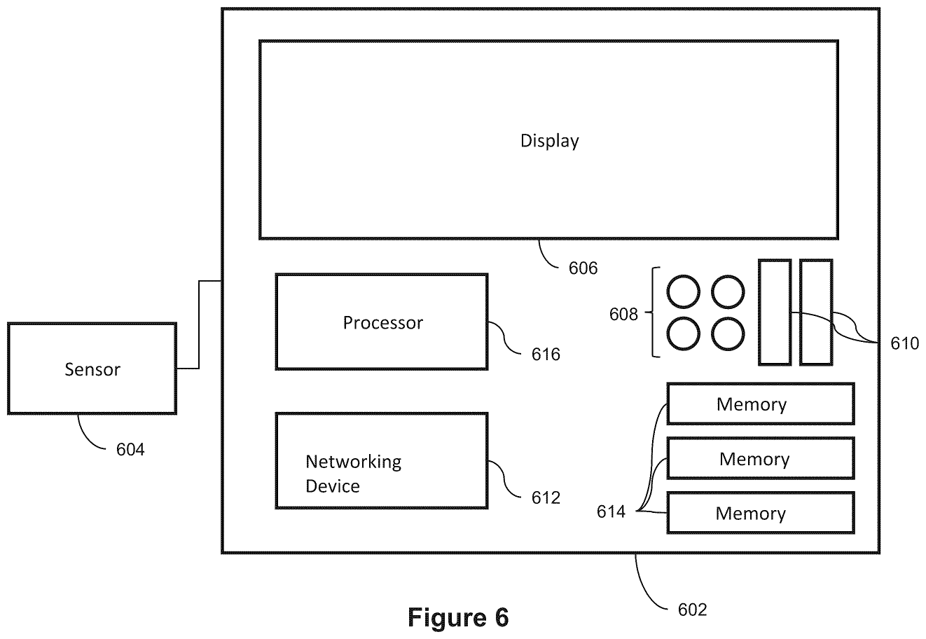

Turning to FIG. 6, processes to construct a capnogram can be executed by a medical monitoring system 602. The medical monitoring system can be a computer, a hemodynamic monitoring system, an electrocardiogramaystem, or any other medical monitoring system. Medical monitoring system can be in connection with a sensor 604, which can provide physiological signals to be analyzes the medical monitoring system. Medical monitoring system 602 can include one or more of a display 606, input controls 608, input and/or output ports 610, networking device 612, memory 614, computational processing system 616, and any other relevant component for a medical monitoring system 602. Display 606 can provide an output of a capnogram, physiological waveform and/or other vital signs, (e.g., heart rate, blood pressure, SₐO₂/SₚO₂ and/or any other relevant information for a medical professional). Display 606 can also provide alerts to indicate that closer monitoring and/or a medical intervention should be performed. Additionally, display 606 can provide menus, options, and/or other selections to provide input into medical monitoring system 602. Such input can be provided via input controls 608, and input controls 608 can include a keypad, keyboard, point and click device, knobs, buttons, and/or any other relevant input control. Alternatively, display 606 and input controls 608 can be combined as a touch screen device, which can be used via a finger, stylus, and/or any other input device. Input and/or output ports 610 can be used to connect medical monitoring system 602 to peripheral devices (e.g., sensor 604), storage devices, and/or any other device for providing input and/or output to medical monitoring system 602. In some implementations of medical monitoring system 602, a networking device 612 can be used to provide communication (wired, wireless, etc.) to another device, such as through a network, near-field communication, Bluetooth, infrared, radio frequency, and/or any other suitable communication system or protocol. Such systems can be beneficial for receiving data, information, or input from another computing device and/or for transmitting data, information, or output to another device.

Generally, medical monitoring device 602 can be implemented as a specialized monitoring device (e.g., hemodynamic monitor), but it can also be implemented as a more common computing device, such as a desktop computer, tablet, mobile device, laptop computer, notebook computer, and/or server system.

Memory 614 can include non-volatile memory and/or volatile memory, and the processor 616 is a processor, microprocessor, controller, or a combination of processors, microprocessor, and/or controllers that performs instructions stored in memory 614. Such instructions stored in the memory 614, when executed by the processor 616, can direct the processor 616, to perform one or more features, functions, methods, and/or steps as described herein. In some implementations, memory 614 includes an application for performing capnogram construction from a physiological waveform. Any input information or data can be stored in the memory 614—either the same memory or another memory. In accordance with various other embodiments, the medical monitoring system 602 may have hardware and/or firmware that can include the instructions and/or perform these processes.

Sensor 604 can include one or more of invasive and non-invasive components capable of capturing a physiological signals, such that a physiological waveform can be obtained or derived. Sensors that can be utilized to capture physiological signals include a blood pressure transducer catheter, a blood pressure cuff, an ultrasound transducer, MRI scanner, ECG leads, PPG, and any other relevant sensor for capturing physiological signals. Sensor 604 can be in communication with medical monitoring system 602 such that physiological signal data can be transmitted. Such communication can include electrical, pneumatic, and/or any other relevant communication.

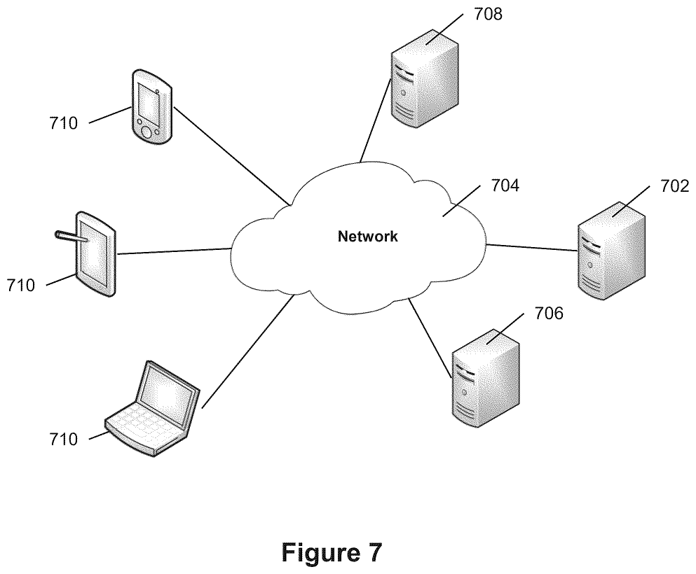

Turning to FIG. 7, an example of distributed medical monitoring system for constructing a capnogram is illustrated in which computing processing and/or other components may rely on remote systems (e.g., cloud system). For example, a distributed medical monitoring system may be useful in a situation in which computing power is not possible at a local level, and a remote computational processing system (e.g., server) performs one or more features, functions, methods, and/or steps described herein. In another example, a distributed medical monitoring system may be useful for remote communication with medical personnel that are not present at the bedside of the patient and/or medical monitor. A computing device 702 (e.g., server) can be connected to a network 704 (wired and/or wireless), where it can receive inputs from one or more computing devices, including data from a records database or repository 706, data provided from a laboratory computing device 708, and/or any other relevant information from one or more other remote devices 710. Once computing device 702 performs one or more features, functions, methods, and/or steps described herein, any outputs can be transmitted to one or more computing devices 706, 708, 710 for entering into records.

While FIGS. 6 and 7 illustrate various systems and computing devices capable of performing steps, processes, and/or functions described herein, additional implementations include the storage of such instructions on machine-readable media, including non-transitory media that is capable of directing a processing device (e.g., processor, microprocessor, etc.) of executing the various methods can be steps, processes, and/or functions described herein. The instructions for the processes can be stored in any of a variety of non-transitory computer readable media appropriate to a specific application.

### EXAMPLES

The systems and methods for constructing a capnogram as described herein can be implemented in a variety of medical procedures. Any medical procedure that would involve obtaining or deriving a physiological waveform can utilize the described systems and processes for constructing a capnogram. In addition, any medical procedure that would benefit from obtaining a capnogram can utilize a sensor to capture physiological signals such that a physiological waveform can be obtained or derived can utilize the described systems and processes for constructing a capnogram. Accordingly, the described systems and processes for constructing a capnogram can be utilized in a variety of surgical procedures, medical procedures involving anesthesia, cardiological monitoring procedures, cancer treatment procedures, endoscopic procedures, labor and birth procedures, miscarriage and abortion procedures, invasive diagnostic procedures, emergency/urgent care procedures, and intensive care procedures.

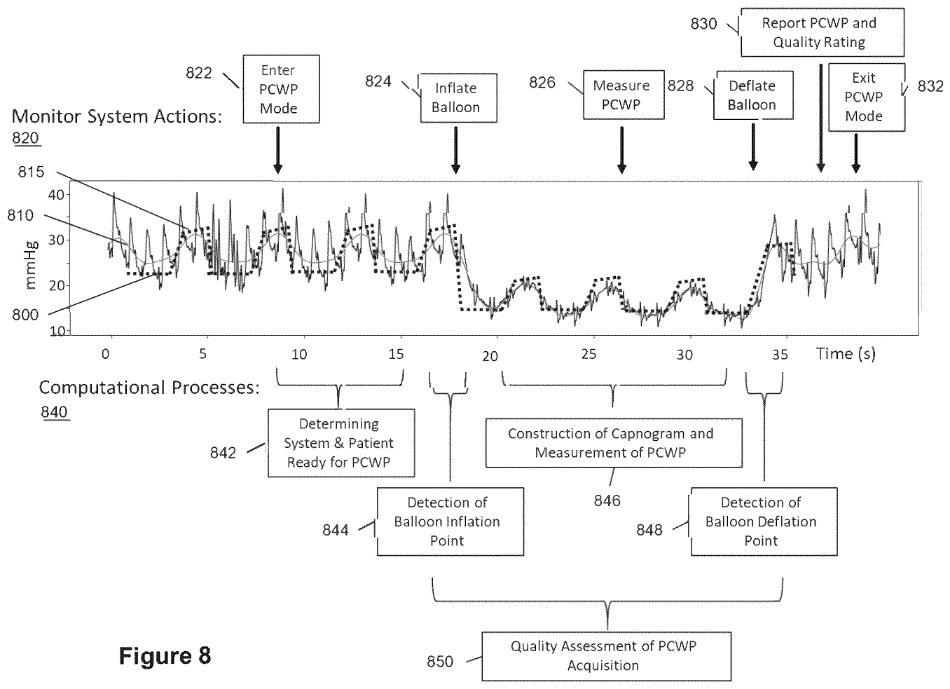

In one example, systems and methods for constructing a capnogram can be utilized in a procedure for measuring pulmonary capillary wedge pressure. Provided in FIG. 8 is an example of a multi-step procedure for computer-assisted measure of pulmonary capillary wedge pressure (PCWP). A hemodynamic monitoring system can perform one or more of the set of actions and processes, in any combination thereof. In some implementations, the set of actions and processes performed by the hemodynamic monitoring system enable automated acquisition and/or assessment of a PCWP measurement.

A hemodynamic monitoring system can comprise a pulmonary artery catheter (e.g., Swan-Ganz catheter) and computational processing system. The pulmonary artery catheter can comprise one or more sensors such as (for example) a pressure transducer, thermal sensor (e.g., for measurement of cardiac output via thermodilution), and a fiber optics (e.g., photometric or other optical measurements). The pulmonary artery catheter can also comprise a balloon near the distal end for performing the wedge method within the pulmonary artery. Utilizing the pulmonary artery catheter, the hemodynamic monitoring system can measure blood pressure, cardiac output, and various other hemodynamic measurements in real time. The monitoring system can further comprise one or more computational programs to assist in monitoring hemodynamic parameters, performing various measurements (e.g., PCWP), and/or derive various measurements (e.g., capnogram construction). To perform the computational tasks, the monitoring system can include a processor, memory, display, one or more computational programs stored in the memory and run by the processor, and one or more ports for connecting the various sensors with the system.

As depicted in FIG. 8, a hemodynamic monitoring system can perform a set of actions (820) and a set of computational processes (840). A pulmonary artery catheter can be positioned within the pulmonary artery and acquiring pressure waveform (800). To measure PCWP, the monitoring system can enter PCWP Mode (822) to initiate the set of actions and computational processes. Upon entering PCWP Mode, the monitoring system can determine whether the system and patient are ready PCWP (822). Upon determining the system and patient are ready for PCWP measurement, in some implementations, the monitoring system can provide a signal to the clinician that the balloon inflation can commence. In some implementations, upon a ready determination, the monitoring system automatically begins the process of balloon inflation.

The hemodynamic monitoring system inflates the balloon (824) at the distal end of the catheter, allowing the catheter to migrate to the wedge position. By assessing hemodynamic parameters derived from the pressure waveform, the hemodynamic system can detect the balloon inflation point (844), giving an indication that the catheter is wedge position.

Upon detection of balloon inflation, in some implementations, the hemodynamic monitoring system can perform one or more computational processes to construct a capnogram (846) from the blood pressure waveform (800) and without direct measurement of gaseous components. Respiratory cycles (810) are shown along with the blood pressure waveform (800). In some implementations, respiratory cycles are extracted utilizing the blood pressure waveform. In some implementations, respiratory cycles are extracted using a lowpass filter cutoff with cutoff below the patient heartrate. In some implementations, respiratory cycles are extracted using a lowpass filter cutoff with cutoff at or below 0.5 Hz. The various lowpass filter cutoffs that are described herein can be utilized in this example. A capnogram can be constructed based on the extracted respiratory waveform by selecting mathematical bases of capnogram waveform morphologies as described herein. In some implementations, to prevent overfitting, a sparse number of selected representative mathematical bases are selected.

The hemodynamic monitoring system can measure PCWP (826) utilizing a computational process for measurement of PCWP (846). In some implementations, PCWP is measured in accordance with the constructed capnogram. In certain implementations, PCWP is the pressure measured at the end-tidal carbon dioxide (ETCO₂) of the constructed capnogram. In some implementations, PCWP is the average pressure measured for a number of capnogram cycles during wedging (e.g., 1, 2, 3, or 4 capnogram cycles). In some implementations, PCWP is the average pressure measured for a period of time during wedging (e.g., between 2 and 20 seconds).

Upon completion of the PCWP measurement, the hemodynamic monitoring system deflates the balloon (828) at the distal end of the catheter, allowing the catheter retract back into the pulmonary artery. In a similar manner to detecting balloon inflation, the monitoring system can include a computational process for detection of the balloon deflation point (848).

The hemodynamic monitoring system can perform a quality assessment of PCWP acquisition (850) by analyzing hemodynamic data between the balloon inflation point and the balloon deflation point. In some implementations, quality of PCWP acquisition can be performed by analyzing the pressure waveform and/or capnogram waveform. Quality ratings can be quantitative (e.g., score on a scale of 0-100) or categorical (e.g., “good” versus “bad,” “adequate” versus “poor,” etc.). In some implementations, thresholds are utilized to determine whether a quality score is low/high or “bad/good”. Thresholds can be based on clinical data.

The hemodynamic monitoring system can report the acquired PCWP and/or the quality rating of the PCWP measurement. In some implementations, the acquired PCWP and/or the quality of the PCWP measurement is displayed on a display screen of the monitoring system. In some implementations, the quality rating is saved in a memory or transmitted to another computational device for storage or downstream analysis. In some implementations, a low or “bad” quality score is utilized to inform the clinician that repeating a PCWP measurement is recommended. In some implementations, a low or “bad” quality score is utilized to automatically repeat a PCWP measurement. Upon completion of the PCWP measurement (or a repeat of the PCWP measurement), the monitoring system can exit PCWP mode (832).

For more details on computer-assisted PCWP measurements, see International Pat. Appl. No. PCT/US2023/027678, the disclosure of which is incorporated by reference in its entirety.

### Examples

Example 1. A computational method to construct a capnogram from a physiological waveform, comprising: obtaining, utilizing a computational processing system, a physiological waveform; filtering, utilizing the computational processing system, the physiological waveform to yield a respiration waveform; and constructing, using the computational processing system, a capnogram based on the respiration waveform.

Example 2. The method of example 1, wherein obtaining a physiological waveform comprises capturing physiological signals from a sensor, wherein the physiological signals are utilized to generate the physiological waveform via the computational processing system.

Example 3. The method of example 2, wherein the sensor is one of: a blood pressure transducer catheter, a blood pressure cuff, an ultrasound transducer, a MRI scanner, ECG leads, or a PPG.

Example 4. The method of example 2 or 3, wherein the sensor is an invasive sensor.

Example 5. The method of example 2 or 3, wherein the sensor is a noninvasive sensor.

Example 6. The method of any one of examples 2-5, wherein filtering the physiological waveform and constructing a capnogram are performed while obtaining a physiological waveform.

Example 7. The method of any one of examples 2-6, wherein a medical monitoring system comprises or is in communication with the computational processing system and the sensor.

Example 8. The method of any one of examples 2-7, wherein obtaining a physiological waveform comprises capturing physiological signals from multiple sensors from multiple locations; wherein the physiological signals from the multiple sensors are combined to generate the physiological waveform via the computational processing system.

Example 9. The method of any one of examples 1-8, wherein the physiological waveform is a blood pressure waveform, a signal proportional to a blood pressure waveform, or a signal derived from a blood pressure waveform.

Example 10. The method of any one of examples 1-8, wherein the physiological waveform is a blood flow waveform, a signal proportional to a blood flow waveform, or a signal derived from a blood flow waveform.

Example 11. The method of any one of examples 1-8, wherein the physiological waveform is an electrocardiogram, a signal proportional to an electrocardiogram, or a signal derived from an electrocardiogram.

Example 12. The method of any one of examples 1-8, wherein the physiological waveform is a plethysmogram, a signal proportional to a plethysmogram, or a signal derived from a plethysmogram.

Example 13. The method of any one of examples 1-12, wherein filtering the physiological waveform comprises using a lowpass filter.

Example 14. The method of example 13, wherein the lowpass filter has a cutoff frequency that is below a heart rate within the physiological waveform.

Example 15. The method of example 13 or 14, wherein filtering the physiological waveform comprises: determining, using the computational processing system, a heart rate from the physiological waveform; and setting, using the computational processing system, a lowpass filter cutoff at a frequency less than the heart rate determined from the physiological waveform.

Example 16. The method of example 13, 14, or 15, wherein the lowpass filter has a cutoff frequency at or below 1 Hz.

Example 17. The method of example 16, wherein the lowpass filter has a cutoff frequency at or below 0.5 Hz.

Example 18. The method of any one of examples 1-17, wherein constructing a capnography waveform comprises: selecting, using the computational processing system, mathematical bases of capnogram waveform morphologies; and constructing, using the computational processing system, a capnography waveform cycle using the selected mathematical bases and the respiration waveform.

Example 19. The method of example 18, wherein the mathematical bases are selected from a database of mathematical bases that represent capnogram waveform morphologies.

Example 20. The method of example 18 or 19, wherein selecting mathematical bases is based on clinical data, patient demographic data, or hemodynamic features.

Example 21. The method of example 18, 19, or 20, wherein a sparse number of mathematical bases is selected.

Example 22. The method of example 21, wherein the number of mathematical bases to be selected can be determined using an equation to identify the bases with a minimum weight or weights.

Example 23. The method of example 22, wherein a weighting vector is solved to be sparse using L1 regularization or L2 regularization.

Example 24. The method of example 22 or 23, wherein selecting the mathematical bases comprises:

$${min}_{w} ( {\sum }_{n = 1}^{N} {w}_{n} {Basis}_{n} - respiration signal ) ,$$

wherein wₙ is a weight of Basisₙ.

Example 25. The method of example 22, 23, or 24, wherein the mathematical bases and respiration waveform are dynamically time warped (DTW) prior to determining the weights for the mathematical bases.

Example 26. The method of any one of examples 18-25, wherein constructing the capnography waveform cycle comprises: Σₙ₌₁^{N} wₙ Basisₙ.

Example 27. The method of any one of examples 18-26, wherein selecting mathematical bases of capnogram waveform morphologies and constructing the capnography waveform cycle is repeated for each respiration cycle.

Example 28. The method of any one of examples 1-27 further comprising displaying the capnogram on a display screen, wherein the computational processing system is in connection with the display screen.

Example 29. The method of any one of examples 1-28 further comprising generating, using the computational processing system, an alert based on the capnogram.

Example 30. The method of example 29, wherein the alert is to indicate that closer monitoring or a medical intervention should be performed.

Example 31. A medical monitoring system for constructing a capnogram, comprising: a sensor, a computational processing system, and a set of instructions stored in memory or in non-transitory media, wherein the sensor is capable of capturing physiological signals, and the sensor is in connection with the computational processing system such that physiological signal data captured by the sensor can be transmitted to the computational processing system, wherein the set of instructions direct the computational processing system to: receive a physiological waveform from the physiological sensor data derived from the sensor; filter the physiological waveform to yield a respiration waveform; and construct a capnogram based on the respiration waveform.

Example 32. The system of example 31, wherein the capnogram is constructed as the physiological sensor data is being received.

Example 33. The system of example 31 or 32, wherein the sensor is one of: a blood pressure transducer catheter, a blood pressure cuff, an ultrasound transducer, a MRI scanner, ECG leads, or a PPG.

Example 34. The system of example 31, 32, or 33, wherein the sensor is an invasive sensor.

Example 35. The system of example 31, 32, or 33, wherein the sensor is a noninvasive sensor.

Example 36. The system of any one of examples 31-35 wherein the set of instructions further direct the computational processing system to: receive physiological sensor data from the sensor; and generate the physiological waveform from the physiological sensor data received from the data sensor.

Example 37. The system of any one of examples 31-36 comprising two or more sensors, wherein the set of instructions further direct the computational processing system to: receive physiological sensor data from each of the two or more sensors; and generate the physiological waveform from the physiological sensor data received from the data two or more sensors; wherein the physiological sensor data from each sensor is combined to generate the physiological waveform.

Example 38. The system of any one of examples 31-37, wherein the physiological waveform is a blood pressure waveform, a signal proportional to a blood pressure waveform, or a signal derived from a blood pressure waveform.

Example 39. The system of any one of examples 31-37, wherein the physiological waveform is a blood flow waveform, a signal proportional to a blood flow waveform, or a signal derived from a blood flow waveform.

Example 40. The system of any one of examples 31-37, wherein the physiological waveform is an electrocardiogram, a signal proportional to an electrocardiogram, or a signal derived from an electrocardiogram.

Example 41. The system of any one of examples 31-37, wherein the physiological waveform is a plethysmogram, a signal proportional to a plethysmogram, or a signal derived from a plethysmogram.

Example 42. The system of any one of examples 31-41, wherein a lowpass filter is used to filter the physiological waveform.

Example 43. The system of example 42, wherein the lowpass filter has a cutoff frequency that is below a heart rate within the physiological waveform.

Example 44. The system of example 42 or 43, wherein the set of instructions further direct the computational processing system to: determine a heart rate from the physiological sensor data; and set a lowpass filter cutoff at a frequency less than the heart rate determined from the physiological signals.

Example 45. The system of example 42, 43, or 44, wherein the lowpass filter has a cutoff frequency at or below 1 Hz.

Example 46. The system of example 45, wherein the lowpass filter has a cutoff frequency at or below 0.5 Hz.

Example 47. The system of any one of examples 31-46, wherein the set of instructions further direct the computational processing system to: select mathematical bases of capnogram waveform morphologies; and construct a capnography waveform cycle using the selected mathematical bases and the respiration waveform.

Example 48. The system of example 47, wherein a database of mathematical bases that represent capnogram waveform morphologies is utilized for selecting the mathematical bases.

Example 49. The system of example 48, wherein the database of mathematical bases that represent capnogram waveform morphologies is stored in the memory or in the non-transitory media.

Example 50. The system of example 47, 38, or 49, wherein selecting mathematical bases is based on clinical data, patient demographic data, or hemodynamic features.

Example 51. The system of any one of examples 47-50, wherein a sparse number of mathematical bases is selected.

Example 52. The system of example 51, wherein the number of mathematical bases to be selected can be determined using an equation to identify the bases with a minimum weight or weights.

Example 53. The system of example 52, wherein a weighting vector is solved to be sparse using L1 regularization or L2 regularization.

Example 54. The system of example 52 or 53, wherein:

$${min}_{w} ( {\sum }_{n = 1}^{N} {w}_{n} {Basis}_{n} - respiration signal )$$

is utilized to select the mathematical bases; wherein wₙ is a weight of Basisₙ.

Example 55. The system of example 52, 53, or 54, wherein the mathematical bases and respiration waveform are dynamically time warped (DTW) prior to determining the weights for the mathematical bases.

Example 56. The system of any one of examples 47-55, wherein: Σₙ₌₁^{N}wₙ Basisₙ is utilized to construct the capnography waveform cycle.

Example 57. The system of any one of examples 47-56, wherein selection of the mathematical bases of capnogram waveform morphologies and construction of the capnography waveform cycle are repeated for each respiration cycle.

Example 58. The system of any one of examples 31-57 further comprising a display screen in connection with the computational processing system; wherein the set of instructions further direct the computational processing system to display the capnogram on a display screen.

Example 59. The system of any one of examples 31-58, wherein the set of instructions further direct the computational processing system to generate an alert based on the capnogram.

Example 60. The system of example 59, wherein the alert is to indicate that closer monitoring or a medical intervention should be performed.

Example 61. The system of any one of examples 31-60, wherein the medical monitoring system is a hemodynamic monitoring system or an electrocardiogramaystem.

Example 62. A computational method for performing a pulmonary capillary wedge pressure (PCWP) measurement of an individual, comprising: acquiring a blood pressure waveform of an individual, wherein the waveform comprises a blood pressure measurement acquired from a pulmonary artery position and a blood pressure measurement acquired from a wedge position, wherein the blood pressure waveform is acquired using a pulmonary artery catheter; constructing, using a computational processing system and the blood pressure waveform, a capnogram of the individual; and determining, using a computational processing system, a PCWP measurement from the blood pressure measurement from the wedge position, wherein the PCWP measurement is determined based on the capnogram of the individual.

Example 63. The method of example 62, wherein the determining of the PCWP comprises determining a blood pressure measurement from the wedge position at an end-tidal CO₂ peak of the capnogram.

Example 64. The method of example 62 or 63, wherein the determining of the PCWP comprises averaging blood pressure measurement from the wedge position across one or more respiratory cycles of the capnogram.

Example 65. The method of example 62, 63, or 64, wherein the PCWP measurement is generated and the capnogram is constructed as the blood pressure waveform is acquired.

Example 66. The method of example 65 further comprising: displaying the PCWP measurement and the capnogram on a display screen in digital communication with the computational processing system.

Example 67. The method of any one of examples 62-66, wherein the computational processing system and the pulmonary artery catheter are part of a hemodynamic monitoring system.

Example 68. The method of any one of examples 62-67, wherein acquiring the blood pressure waveform comprises: inserting the pulmonary artery catheter into a central vein of the individual, guiding the pulmonary artery catheter to the pulmonary artery; and inflating a balloon at or near a distal end of the pulmonary artery catheter to allow the pulmonary artery catheter to migrate to the wedge position.

Example 69. A hemodynamic monitoring system for performing a pulmonary capillary wedge pressure (PCWP) measurement, comprising: a pulmonary artery catheter configured to acquire blood pressure measurement acquired from a pulmonary artery position and a blood pressure measurement acquired from a wedge position, wherein the pulmonary artery catheter comprises a balloon that is inflatable; and a computational processing system in connection with the pulmonary artery catheter, the computational processing system comprising: a processor system; a display screen in digital connection with the processor system; and a memory system comprising one or more applications that can direct the processor system to: acquire a blood pressure measurement in a pulmonary artery position; inflate the balloon, wherein inflating the balloon allows the catheter to migrate to a wedge position; acquire a blood pressure measurement in the wedge position; generate a blood pressure waveform from blood pressure measurement acquired from a pulmonary artery position and a blood pressure measurement acquired from a wedge position; construct a capnogram using the blood pressure waveform; determine a PCWP measurement from the blood pressure measurement from the wedge position, wherein the PCWP measurement is determined based on the capnogram; and display one or more of: the blood pressure waveform, the PCWP measurement, or the capnogram on the display screen.

Example 70. The system of example 69, wherein the determining of the PCWP comprises determining a blood pressure measurement from the wedge position at an end-tidal CO₂ peak of the capnogram.

Example 71. The system of example 69 or 70, wherein the determining of the PCWP comprises averaging blood pressure measurement from the wedge position across one or more respiratory cycles of the capnogram.

Example 72. The system of example 69, 70, or 71, wherein the blood pressure waveform is generated and displayed on the display screen in real time.

Example 73. The system of any one of examples 69-72, wherein the PCWP measurement is determined and displayed on the display screen in real time.

Example 74. The system of any one of examples 69-73, wherein the capnogram is constructed and displayed on the display screen as the blood pressure waveform is generated.

What is claimed is:

1. A computational method to construct a capnogram from a physiological waveform, comprising:
obtaining, utilizing a computational processing system, the physiological waveform;
filtering, utilizing the computational processing system, the physiological waveform to yield a respiration waveform; and
constructing, using the computational processing system, the capnogram based on the respiration waveform.

2. The method of claim 1, wherein obtaining the physiological waveform comprises capturing physiological signals from a sensor, wherein the physiological signals are utilized to generate the physiological waveform via the computational processing system.

3. The method of claim 2, wherein the sensor is one of: a blood pressure transducer catheter, a blood pressure cuff, an ultrasound transducer, an MRI scanner, ECG leads, or a PPG.

4. The method of claim 2, wherein filtering the physiological waveform and constructing the capnogram are performed while obtaining the physiological waveform.

5. The method of claim 2, wherein a medical monitoring system comprises or is in communication with the computational processing system and the sensor.

6. The method of claim 1, wherein obtaining the physiological waveform comprises capturing physiological signals from multiple sensors from multiple locations; wherein the physiological signals from the multiple sensors are combined to generate the physiological waveform via the computational processing system.

7. The method of claim 1, wherein the physiological waveform comprises:
a blood pressure waveform, a signal proportional to a blood pressure waveform, or a signal derived from a blood pressure waveform;
a blood flow waveform, a signal proportional to a blood flow waveform, or a signal derived from a blood flow waveform;
an electrocardiogram, a signal proportional to an electrocardiogram, or a signal derived from an electrocardiogram; or
a plethysmogram, a signal proportional to a plethysmogram, or a signal derived from a plethysmogram.

8. The method of claim 1, wherein filtering the physiological waveform comprises using a lowpass filter.

9. The method of claim 8, wherein the lowpass filter has a cutoff frequency that is below a heart rate within the physiological waveform.

10. The method of claim 8, wherein filtering the physiological waveform comprises:
determining, using the computational processing system, a heart rate from the physiological waveform; and
setting, using the computational processing system, a lowpass filter cutoff at a frequency less than the heart rate determined from the physiological waveform.

11. The method of claim 1, wherein constructing the capnography waveform comprises:
selecting, using the computational processing system, mathematical bases of capnogram waveform morphologies; and
constructing, using the computational processing system, a capnography waveform cycle using the selected mathematical bases and the respiration waveform.

12. The method of claim 11, wherein the mathematical bases are selected from a database of mathematical bases that represent capnogram waveform morphologies.

13. The method of claim 11, wherein selecting mathematical bases is based on clinical data, patient demographic data, or hemodynamic features.

14. The method of claim 11, wherein a sparse number of mathematical bases is selected.

15. The method of claim 14, wherein a number of mathematical bases selected is determined using an equation to identify the bases with a minimum weight or weights.

16. The method of claim 15, wherein selecting the mathematical bases is performed according to the following formula:
$${min}_{w} ( {\sum }_{n = 1}^{N} {w}_{n} {Basis}_{n} - respiration signal )$$
wherein wₙ is a weight of mathematical Basisₙ.

17. The method of claim 15, wherein the mathematical bases and respiration waveform are dynamically time warped (DTW) prior to determining the weights for the mathematical bases.

18. The method of claim 11, wherein constructing the capnography waveform cycle comprises:
Σₙ₌₁^{N}wₙBasisₙ
wherein wₙ is a weight of mathematical Basisₙ.

19. The method of claim 11, wherein selecting mathematical bases of capnogram waveform morphologies and constructing the capnography waveform cycle is repeated for each respiration cycle.

20. The method of claim 1, further comprising displaying the capnogram on a display screen, wherein the computational processing system is in connection with the display screen.

21. A medical monitoring system for constructing a capnogram, comprising:
a sensor, a computational processing system, and a set of instructions stored in memory or in non-transitory media, wherein the sensor is capable of capturing physiological signals, and the sensor is in connection with the computational processing system such that physiological signal data captured by the sensor can be transmitted to the computational processing system, wherein the set of instructions direct the computational processing system to:
receive a physiological waveform from the physiological sensor data derived from the sensor;
filter the physiological waveform to yield a respiration waveform; and
construct a capnogram based on the respiration waveform.

22. The system of claim 21, wherein the capnogram is constructed as the physiological sensor data is being received.

23. The system of claim 21, wherein the sensor is one of: a blood pressure transducer catheter, a blood pressure cuff, an ultrasound transducer, an MRI scanner, ECG leads, or a PPG.

24. The system of any claim 21, wherein the set of instructions further direct the computational processing system to:
receive physiological sensor data from the sensor; and
generate the physiological waveform from the physiological sensor data received from the data sensor.

25. The system of claim 21 comprising two or more sensors, wherein the set of instructions further direct the computational processing system to:
receive physiological sensor data from each of the two or more sensors; and
generate the physiological waveform from the physiological sensor data received from the data two or more sensors; wherein the physiological sensor data from each sensor is combined to generate the physiological waveform.

26. The system of claim 21, wherein the physiological waveform is:
a blood pressure waveform, a signal proportional to a blood pressure waveform, or a signal derived from a blood pressure waveform;
a blood flow waveform, a signal proportional to a blood flow waveform, or a signal derived from a blood flow waveform;
an electrocardiogram, a signal proportional to an electrocardiogram, or a signal derived from an electrocardiogram; or
a plethysmogram, a signal proportional to a plethysmogram, or a signal derived from a plethysmogram.

27. The system of claim 21, wherein a lowpass filter is used to filter the physiological waveform.

28. The system of claim 27, wherein the lowpass filter has a cutoff frequency that is below a heart rate within the physiological waveform.

29. The system of claim 27, wherein the set of instructions further direct the computational processing system to:
determine a heart rate from the physiological sensor data; and
set a lowpass filter cutoff at a frequency less than the heart rate determined from the physiological signals.

30. The system of claim 21, wherein the set of instructions further direct the computational processing system to:
select mathematical bases of capnogram waveform morphologies; and
construct a capnography waveform cycle using the selected mathematical bases and the respiration waveform.

31. The system of claim 30, wherein a database of mathematical bases that represent capnogram waveform morphologies is utilized for selecting the mathematical bases.

32. The system of claim 31, wherein the database of mathematical bases that represent capnogram waveform morphologies is stored in the memory or in the non-transitory media.

33. The system of claim 30, wherein selecting mathematical bases is based on clinical data, patient demographic data, or hemodynamic features.

34. The system of claim 30, wherein a sparse number of mathematical bases is selected.

35. The system of claim 34, wherein a number of mathematical bases selected is determined using an equation to identify the bases with a minimum weight or weights.

36. The system of claim 35, wherein selecting the mathematical bases is performed according to the following formula:
$${min}_{w} ( {\sum }_{n = 1}^{N} {w}_{n} {Basis}_{n} - respiration signal )$$
wherein wₙ is a weight of mathematical Basisₙ.

37. The system of claim 35, wherein the mathematical bases and respiration waveform are dynamically time warped (DTW) prior to determining the weights for the mathematical bases.

38. The system of claim 30, wherein the formula:
Σₙ₌₁^{N}wₙBasisₙ
is utilized to construct the capnography waveform cycle, wherein wₙ is a weight of mathematical Basisₙ.

39. The system of claim 30, wherein selection of the mathematical bases of capnogram waveform morphologies and construction of the capnography waveform cycle are repeated for each respiration cycle.

40. The system of claim 21, further comprising a display screen in connection with the computational processing system; wherein the set of instructions further direct the computational processing system to display the capnogram on a display screen.

41. The system of claim 21, wherein the medical monitoring system is a hemodynamic monitoring system or an electrocardiogramaystem.
